(19) United States
(12) Patent Application Publication
(10) Pub. No.: US 2021/0172890 A1
(43) Pub. Date: Jun. 10, 2021

(54) FATIGUE ESTIMATING METHOD, AND METHOD OF CREATING DATABASE FOR FATIGUE ESTIMATION

(71) Applicant: JTEKT CORPORATION, Osaka (JP)

(72) Inventor: Yousuke NAGANO, Yao-shi (JP)

(73) Assignee: JTEKT CORPORATION, Osaka-shi (JP)

(21) Appl. No.: 16/951,370

(22) Filed: Nov. 18, 2020

(30) Foreign Application Priority Data
Dec. 9, 2019 (JP) 2019-221777

(51) Int. Cl. G01N 23/203

(52) U.S. Cl. CPC G01N 23/203; G01N 2223/053; G01N 2223/646

(57) ABSTRACT

A fatigue estimating method of estimating a degree of fatigue of a metallic material includes: estimating a fatigue portion in which fatigue appears in a section of the metallic material, obtaining a crystal grain size of each of a plurality of crystal grains in a measurement area set in the fatigue portion, based on crystal misorientation in the measurement area, obtaining a fatigue portion existence rate indicating an existence proportion of particular crystal grains of which the crystal grain size is within a predetermined numeral range, in the measurement area, and obtaining an estimated degree of fatigue of the metallic material, based on at least one of the fatigue portion existence rate, and a change rate of the fatigue portion existence rate before and after fatigue of the metallic material.

### CROSS-REFERENCE TO RELATED APPLICATION

This application claims priority to Japanese Patent Application No. 2019-221777 filed on Dec. 9, 2019, incorporated herein by reference in its entirety.

### BACKGROUND

### 1. Technical Field

The disclosure relates to a fatigue estimating method of estimating a degree of fatigue of a metallic material, and a method of creating a database for fatigue estimation, for use in the fatigue estimating method.

### 2. Description of Related Art

As a method of estimating a degree of fatigue of a metallic component, it is known to quantitatively measure the amount of strain generated in crystal grains of metal by X-ray diffraction, and estimate the degree of fatigue, based on the amount of strain (see, for example, Japanese Unexamined Patent Application Publication No. 11-344454 (JP 11-344454 A)).

### SUMMARY

The measurement of the amount of strain by X-ray diffraction is advantageous in that the measurement does not require the metallic component to be broken. Meanwhile, the depth of penetration of X-rays when the component is irradiated with the X-rays from the outside is several μm. Thus, when fatigue, such as rolling fatigue in bearing parts, etc., is generated in a relatively deep region inside a surface, X-rays are less likely or unlikely to reach the region where fatigue is generated, which may make it difficult to measure the amount of strain with high accuracy. Therefore, the metallic material may be cut, and its section may be irradiated with X-rays, so that measurement by the X-ray diffraction is performed on the relatively deep region of the metallic material.

Here, when the section of the metallic material is irradiated with the X-rays, the diffraction intensity needs to be increased by expanding the irradiation range of the X-rays, or extending the irradiation time of the X-rays, in an attempt to improve the accuracy of measurement results for improvement of the accuracy in estimation of the fatigue degree. However, if the irradiation range of the X-rays is expanded, portions other than the fatigue portion are more likely to be irradiated with the X-rays, and information on the portions other than the fatigue portion may be included in the measurement results, resulting in reduction of the accuracy of the measurement results. If the irradiation time of the X-rays is extended, the measurement time is extended, and a load on an X-ray diffraction device increases, which may cause a problem in terms of cost.

Thus, if the attempt to increase the diffraction intensity is made so as to improve the accuracy of measurement values when the degree of fatigue is estimated from the section of the metallic material by X-ray diffraction, the accuracy of the measurement values may be reduced to the contrary, or the cost may be increased. Thus, there is a problem that it is difficult to improve the accuracy in estimation of the fatigue degree.

The disclosure provides a technology that can improve the accuracy in estimating the degree of fatigue of a metallic material.

For estimation of the fatigue degree of a metallic material, the inventor of the present disclosure focused on analysis using electron backscatter diffraction (EBSD) with which crystal analysis of metallic materials can be conducted, like the X-ray diffraction method. When the crystal grain size of each of a plurality of crystal grains in a measurement area of a metallic material was measured with the EBSD, as one example of the crystal analysis, it was found that an existence proportion of crystal grains of which the crystal grain sizes are within a predetermined numerical range, in the measurement area, has a good correlative relationship with the degree of fatigue of the metallic material. The inventor completed this disclosure based on this finding.

Namely, this disclosure is concerned with a fatigue estimating method of estimating the degree of fatigue of a metallic material, and the method includes the steps of: estimating a fatigue portion in which fatigue appears in a section of the metallic material, obtaining a crystal grain size of each of a plurality of crystal grains in a fatigue portion measurement area set in the fatigue portion, based on crystal misorientation in the fatigue portion measurement area, obtaining a fatigue portion existence rate indicating an existence proportion of particular crystal grains of which the crystal grain size is within a predetermined numeral range, in the fatigue portion measurement area, and obtaining an estimated degree of fatigue of the metallic material, based on at least one of the fatigue portion existence rate, and a change rate of the fatigue portion existence rate before and after fatigue of the metallic material.

According to the fatigue estimating method as described above, the crystal grain size is obtained based on crystal misorientation measured using the EBSD, so as to obtain the estimated degree of fatigue. Thus, the crystal grain size in a more minute range than that of the X-ray diffraction method can be obtained. Thus, the fatigue portion measurement area can be precisely set for the fatigue portion in which fatigue develops, to permit measurements therein, and the crystal grain size of each of the crystal grains in the fatigue portion measurement area can be obtained with high accuracy. Further, the fatigue portion existence rate obtained based on the crystal grain size, and its change rate, have correlative relationships with the degree of fatigue of the metallic material; therefore, the estimated degree of fatigue of the metallic material can be obtained. Thus, according to the fatigue estimating method as described above, it is possible to obtain the estimated degree of fatigue while assuring high accuracy in the measurement results in the fatigue portion, and improve the estimation accuracy of the fatigue degree.

A portion of the metallic material other than the fatigue portion may maintain a condition before fatigue is generated. Thus, an outside-fatigue-portion existence rate may be considered as a value close to a value of the fatigue portion existence rate before fatigue appears in the fatigue portion. Thus, the fatigue estimating method as described above may further include the steps of: obtaining the crystal grain size of each of the crystal grains in an outside-fatigue-portion measurement area set in a portion of the section of the metallic material other than the fatigue portion, based on the crystal misorientation in the outside-fatigue-portion measurement area, and obtaining an outside-fatigue-portion existence rate indicating the existence proportion of the particular crystal grains in the outside-fatigue-portion measurement area. When the estimated degree of fatigue of the metallic material is obtained, the change rate of the fatigue portion existence rate may be obtained, based on the fatigue portion existence rate, and the outside-fatigue-portion existence rate. In this case, the change rate of the fatigue portion existence rate is obtained, based on the fatigue portion existence rate and the outside-fatigue-portion existence rate; thus, it is possible to obtain the change rate of the fatigue portion existence rate before and after fatigue of the metallic material, without preparing a metallic material before fatigue, in advance.

In the fatigue estimating method as described above, the fatigue portion may be a portion in which rolling fatigue appears, and a distance from a rolling contact surface of the metallic material to the fatigue portion in a depth direction may be equal to or larger than a predetermined value. The portion other than the fatigue portion may be a surface portion between the rolling contact surface of the metallic material and the fatigue portion. When the fatigue generated in the metallic material is rolling fatigue, a metallographic structure of the surface portion between the rolling contact surface and the fatigue portion keeps a condition before the rolling fatigue is generated. Further, when the metallic material is subjected to surface treatment, the influence of the surface treatment on the fatigue portion is also imparted to the surface portion. Thus, the metallographic structure of the fatigue portion before fatigue is substantially identical with that of the surface portion. Thus, the outside-fatigue-portion existence rate in the surface portion can be considered as a value close to a value of the fatigue portion existence rate before fatigue develops in the fatigue portion. Thus, by using the outside-fatigue-portion existence rate in the surface portion, it is possible to obtain the change rate of the fatigue portion existence rate with improved accuracy.

In the fatigue estimating method as described above, the predetermined numerical range may be equal to or smaller than a predetermined set value. In this case, the crystal grains of which the crystal grain sizes are relatively small, and which increase in proportion to fatigue, can be included in the particular crystal grains. Further, the crystal grain size may be a crystal grain area, and the predetermined set value may be equal to or larger than 0.1 μm², and may be equal to or smaller than 2.5 μm². When the set value is smaller than 0.1 μm², the correlative relationship between the change rate of the fatigue portion existence rate and the fatigue degree of the metallic material may be partially discontinuous. Also, when the set value is larger than 2.5 μm², the correlative relationship between the fatigue portion existence rate and the fatigue degree of the metallic material may be partially discontinuous. When the predetermined set value is equal to or larger than 0.1 μm², and is equal to or smaller than 2.5 μm², both the correlation between the change rate of the fatigue portion existence rate and the calculation life ratio, and the correlation between the fatigue portion existence rate and the calculation life ratio can establish good relationships, and the estimated calculation life ratio can be obtained with high accuracy.

The fatigue estimating method may further include creating a database indicating a relationship between at least one of the fatigue portion existence rate, and the change rate of the fatigue portion existence rate, and the degree of fatigue of the metallic material, and the estimated degree of fatigue of the metallic material may be obtained by referring to the database.

This disclosure is also concerned with a method of creating a database for fatigue estimation of a metallic material, for use in the fatigue estimating method as described above. The database is used when the estimated degree of fatigue of the metallic material is obtained, based on at least one of the fatigue portion existence rate, and the change rate of the fatigue portion existence rate. The method includes: obtaining a plurality of test pieces which have different degrees of fatigue, and are formed of the same material as the metallic material, estimating a fatigue portion in which fatigue appears in a section of each of the test pieces, measuring a distribution of misorientations in a fatigue portion measurement area set in the fatigue portion, with respect to each of the test pieces, obtaining a crystal grain size of each of a plurality of crystal grains in the fatigue portion measurement area, based on the distribution of misorientations in the fatigue portion measurement area, with respect to each of the test pieces, obtaining a fatigue portion existence rate indicating an existence proportion of particular crystal grains of which the crystal grain size is within a predetermined numerical range, in the fatigue portion measurement area, with respect to each of the test pieces, and creating the database for fatigue estimation, by associating the degree of fatigue of each of the test pieces, with at least one of the fatigue portion existence rate of each of the test pieces and the change rate of the fatigue portion existence rate of each of the test pieces.

According to the above method, it is possible to obtain the database for use in fatigue estimation, which can further improve the accuracy with which the degree of fatigue is estimated.

According to this disclosure, the estimation accuracy of the degree of fatigue of the metallic material can be further improved.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a flowchart illustrating a method of estimating the degree of fatigue of a metallic material according to one embodiment;

FIG. 2 is a view useful for describing a process of step S1 to step S3 in FIG. 1;

FIG. 3 is a view showing a part of a map representing distribution of crystal misorientations in a measurement area of a fatigue portion;

FIG. 4 is a view showing one example of a correspondence table in which values (classes) of the crystal grain area are associated with the existence proportions (relative frequencies);

FIG. 5A is a view showing the relationship between the crystal grain area before fatigue and the relative frequency (existence proportion);

FIG. 5B is an enlarged view of FIG. 5A in which values of the crystal grain area are within a range from 0 to 1.0 μm²;

FIG. 5C is a view showing the relationship between the crystal grain area in the fatigue portion, and the relative frequency (existence proportion);

FIG. 5D is an enlarged view of FIG. 5C in which values of the crystal grain area are within a range from 0 to 1.0 μm²;

FIG. 6 is a view showing a fatigue database in the form of a graph;

FIG. 7 is a block diagram showing one example of a computing device for obtaining the estimated degree of fatigue;

FIG. 8 is a view showing a histogram obtained when creating the fatigue database shown in FIG. 6, and indicating the relationship between the crystal grain area (class) in a measurement area of a portion other than the fatigue portion, and the existence proportion (relative frequency);

FIG. 9 is a graph which is obtained when creating the fatigue database shown in FIG. 6, and indicates the relationship between the fatigue portion existence rate, and the calculation life ratio;

FIG. 10A is a view showing a fatigue database indicating the relationship between the rate of change of the fatigue portion existence rate, and the calculation life ratio, when a set value is set within a range of 0.05 to 0.50 μm²;

FIG. 10B is a view showing a fatigue database indicating the relationship between the change rate of the fatigue portion existence rate, and the calculation life ratio, when the set value is set within a range of 0.60 to 1.20 μm²;

FIG. 10C is a view showing a fatigue database indicating the relationship between the change rate of the fatigue portion existence rate, and the calculation life ratio, when the set value is set within a range of 1.50 to 5.00 μm²;

FIG. 11A is a view showing a fatigue database indicating the relationship between the fatigue portion existence rate and the calculation life ratio, when the set value is set within a range of 0.05 to 0.50 μm²; and

FIG. 11B is a view showing a fatigue database indicating the relationship between the fatigue portion existence rate and the calculation life ratio, when the set value is set within a range of 0.60 to 1.20 μm²; and

FIG. 11C is a view showing a fatigue database indicating the relationship between the fatigue portion existence rate and the calculation life ratio, when the set value is set within a range of 1.50 to 5.00 μm².

### DETAILED DESCRIPTION OF EMBODIMENTS

Next, a preferred embodiment of the disclosure will be described with reference to the drawings.

About Fatigue Estimation of Metallic Material

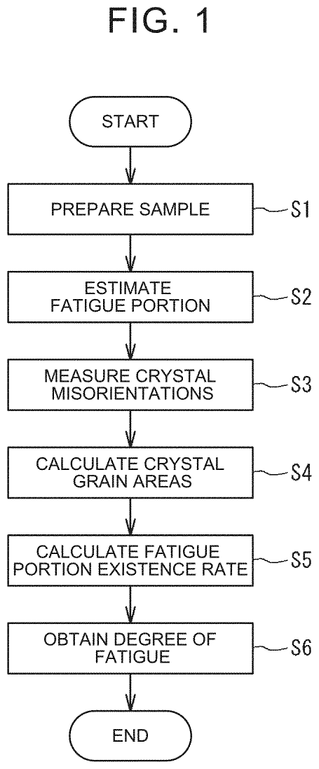

FIG. 1 is a flowchart showing a method of estimating the degree of fatigue of a metallic material according to the embodiment. In this method, initially, as a preparation step when estimating the degree of fatigue, sample preparation is performed for a metallic material as a sample of which the degree of fatigue is to be estimated (step S1). Then, using the metallic material thus prepared, a portion (fatigue portion) in which fatigue develops in a section of the metallic material is estimated (step S2). Then, a measurement area is set in the estimated fatigue portion, and measurement of crystal misorientations using the EBSD (electron backscatter diffraction) is performed on the measurement area (step S3). Then, respective crystal grain sizes of a plurality of crystal grains in the measurement area are obtained, based on the measurement result of the crystal misorientations (step S4). In this embodiment, the crystal grain area is obtained, as the crystal grain size of each of the crystal grains. Then, based on the crystal grain areas obtained in step S4, the fatigue portion existence rate is obtained which indicates the existence proportion of particular crystal grains of which the crystal grain areas are within a predetermined numerical range, in the measurement area (step S5). Then, the rate of change of the fatigue portion existence rate in the fatigue portion is obtained, based on the fatigue portion existence rate obtained in step S5, and the estimated degree of fatigue of the metallic material is obtained, based on the rate of change (step S6).

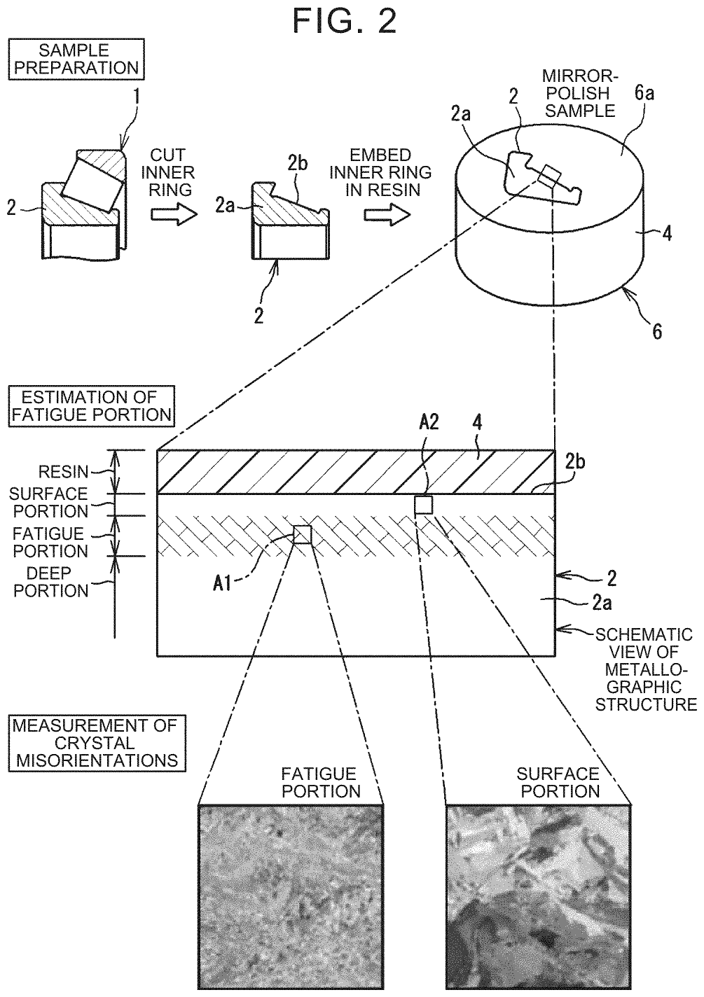

Referring to FIG. 2, the process of step S1 to step S3 in FIG. 1 will be described. In the following, the case where an inner ring of a tapered roller bearing is used, as the metallic material of which the fatigue degree is to be estimated, will be described. In FIG. 2, in the step (step S1 in FIG. 1) of preparing a sample, a measurement sample 6 is obtained by cutting an inner ring 2 of a tapered roller bearing 1 with a high-speed cutter, or the like, and embedding the inner ring 2 in the form of a cut piece in resin 4. The inner ring 2 is embedded in the resin 4 in a condition where a section 2a is exposed from an observation surface 6a of the measurement sample 6. The observation surface 6a to which the section 2a is exposed is mirror-polished, so that the section 2a is mirror-polished. The inner ring 2 is formed using an alloy steel for machine structural use, or carbon steel for machine structural use, for example, and is then subjected to heat treatment.

Then, in FIG. 2, in the step (step S2 in FIG. 1) of estimating the fatigue portion, the mirror-polished section 2a of the inner ring 2 is etched with a given etching fluid, a metallographic structure of the section 2a is observed with a metallographic microscope, or the like, and the fatigue portion is estimated from the observation result of the metallographic structure. FIG. 2 schematically shows the metallographic structure of the section 2a of the inner ring 2 of the tapered roller bearing, which is observed on the observation surface 6a. In the schematic view of the metallographic structure in FIG. 2, the resin 4, and the section 2a of the inner ring 2 embedded in the resin 4 are shown.

For example, in the metallographic structure thus observed, there is a difference in contrast between a region of the fatigue portion and a region other than the fatigue portion. The presence of the fatigue portion and its position can be estimated from the difference in contrast. In the fatigue portion, the size of crystal grains is presumed to be significantly reduced, as compared with the region other than the fatigue region. In a bearing ring of a roller bearing, rolling fatigue develops in a region inside the vicinity of a raceway surface (rolling contact surface) on which a rolling element runs. In the schematic view of the metallographic structure in FIG. 2, in the inner ring 2, a region of a fatigue portion appears in a region inside a raceway surface 2b. Generally, the region of the fatigue portion appears in a range of 50 μm to 200 μm in depth as measured from the surface of the raceway surface 2b. In a surface portion located between the raceway surface 2b and the fatigue portion, and a deep portion located on the inner side of the fatigue portion, the size of crystal grains is not significantly reduced as in the fatigue portion, and no sign of fatigue is observed.

Thus, through observation of the metallographic structure, the fatigue portion as a portion in which fatigue develops compared to its surrounding is estimated, and positional information, etc. of the fatigue portion, including a distance from the raceway surface 2b to the fatigue portion, and the depth, width, etc. of the fatigue portion, are obtained. However, when the roller bearing is used under steady conditions where an applied load is known in advance, the depth and position of the fatigue portion may be estimated by calculation.

Then, in FIG. 2, in the step of measuring the crystal misorientations (step S3 in FIG. 2), the measurement sample 6 of which the metallographic structure has been observed is mirror-polished again, and the mirror-polished measurement sample 6 is set in a scanning electron microscope (SEM), for measurement of crystal misorientations by the EBSD. The crystal misorientation is a relative difference in the crystallographic orientation between adjacent crystal grains, and is represented by a KAM (Kernel Average Misorientation) value, or LOS (Local Orientation Spread) value, for example. To measure crystal misorientations, a measurement area is initially set. When the crystal misorientations in the fatigue portion are measured, a measurement area A1 (see the schematic view of the metallographic structure in FIG. 2) is set in a region of the fatigue portion. By the EBSD, crystal misorientations in the measurement area A1 are measured. The measurement area A1 (fatigue portion measurement area) is a region in which the crystal misorientations in the fatigue portion are measured, and is set as an area where the crystal grain areas of crystal grains in the fatigue portion are to be obtained.

In this embodiment, measurement of crystal misorientations in the surface portion is also conducted, in addition to measurement of the crystal misorientations in the fatigue portion. The crystal misorientations are measured in the surface portion in the same manner as that of measurement of the crystal misorientations in the fatigue portion. More specifically, a measurement area A2 (see the schematic view of the metallographic structure in FIG. 2) is set in a region of the surface portion, and the crystal misorientations are measured in the measurement area A2. The measurement area A2 (outside-fatigue-portion measurement area) is a region in which the crystal misorientations in an area other than the fatigue portion are measured, and is set as an area where the crystal grain areas of crystal grains in the area other than the fatigue portion are to be obtained. Each of the measurement areas A1, A2 is set to the shape of a square of which one side is 30 μm, for example.

Then, as shown in step S4 of FIG. 1, respective crystal grain areas of a plurality of crystal grains are obtained based on the result of measurement of crystal misorientations. Distribution of the crystal misorientations in the measurement area can be obtained from the measurement results of crystal misorientations in the measurement area. In this embodiment, when the misorientation is 15 degrees or larger, it is defined as a crystal grain boundary. Thus, the distribution of crystal misorientations in the measurement area can be divided into two levels, i.e., regions where the misorientation is equal to or larger than 15 degrees, and regions where the misorientation is smaller than 15 degrees, and the distribution of misorientations thus divided into the two levels can be represented by a map.

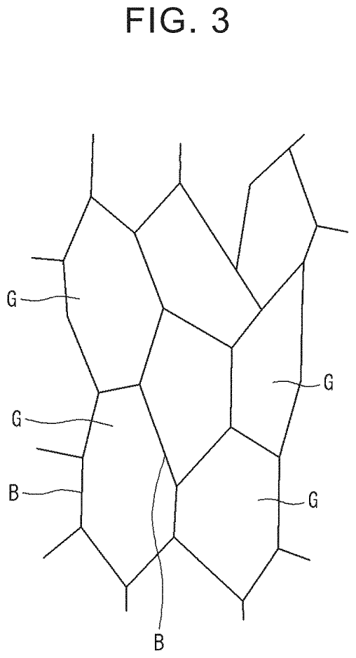

FIG. 3 shows a part of the map representing the distribution of crystal misorientations in the measurement area A1. In FIG. 3, measurement point areas P where the misorientation is 15 degrees or larger are indicated by black (with color), and measurement point areas P where the misorientation is smaller than 15 degrees are indicated by white (with no color). As shown in FIG. 3, a region where the misorientation is 15 degrees or larger appears as a linear portion B. The linear portion B appears in the form of a network. In this embodiment, the linear portion B is defined as a crystal boundary in the measurement area A1, and a region G surrounded by the linear portion B corresponds to a crystal grain, while the area of each region G in the measurement area A1 is obtained as a crystal grain area. In this embodiment, the area of each region G in the measurement area A2 is also obtained as a crystal grain area, like the crystal grain area in the measurement area A1. The area (crystal gran area) of each region G in the measurement area A1 (A2) can be obtained by performing image processing on an image representing distribution of misorientations in the measurement area A1 (A2), for example.

Then, as indicated in step S5 of FIG. 1, the fatigue portion existence rate in the measurement area A1 is obtained. To obtain the fatigue portion existence rate, the crystal grain area of each crystal grain (region G in FIG. 3) in the measurement area A1 is classified as one of fixed numerical ranges, and a frequency distribution is obtained in which the class denotes a value of the crystal grain area thus classified, and the frequency denotes the number of crystal grains in each class. When the crystal grain area is represented by the frequency distribution, the relative frequency indicates the proportion of the number of crystal grains included in each class (value of each crystal grain area), to the number of all crystal grains included in the measurement area A1. Namely, the relative frequency indicates the existence proportion of the crystal grains corresponding to each class, in the measurement area A1.

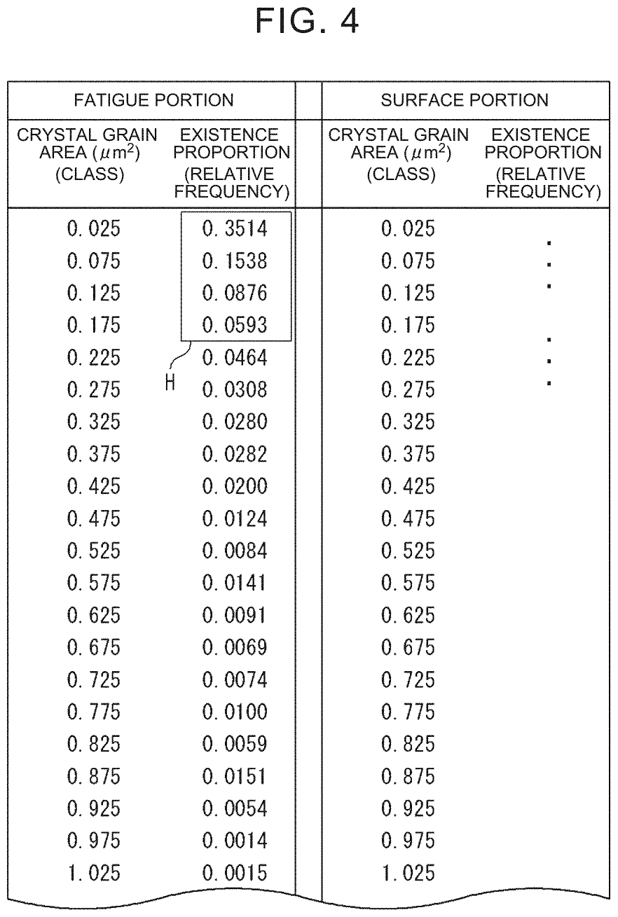

FIG. 4 shows one example of a correspondence table that associates the value (class) of the crystal grain area with the existence proportion (relative frequency). In FIG. 4, the correspondence table includes a frequency distribution in the measurement area A1 of the fatigue portion, and a frequency distribution in the measurement area A2 of the surface portion. Each class is set to a preset numerical width (e.g., 0.050 μm² as a numerical width). In FIG. 4, the class of 0.025 μm² as a value of the crystal grain area is set to a numerical range that is larger than 0.000 μm² and is equal to or smaller than 0.050 μm². Similarly, the class of 0.075 μm² as a value of the crystal grain area is set to a numerical range that is larger than 0.050 μm² and is equal to or smaller than 0.100 μm². Each crystal grain in the measurement area A1 (A2) is classified according to the value of the crystal grain area, to be associated with any of the classes, and is counted as a frequency.

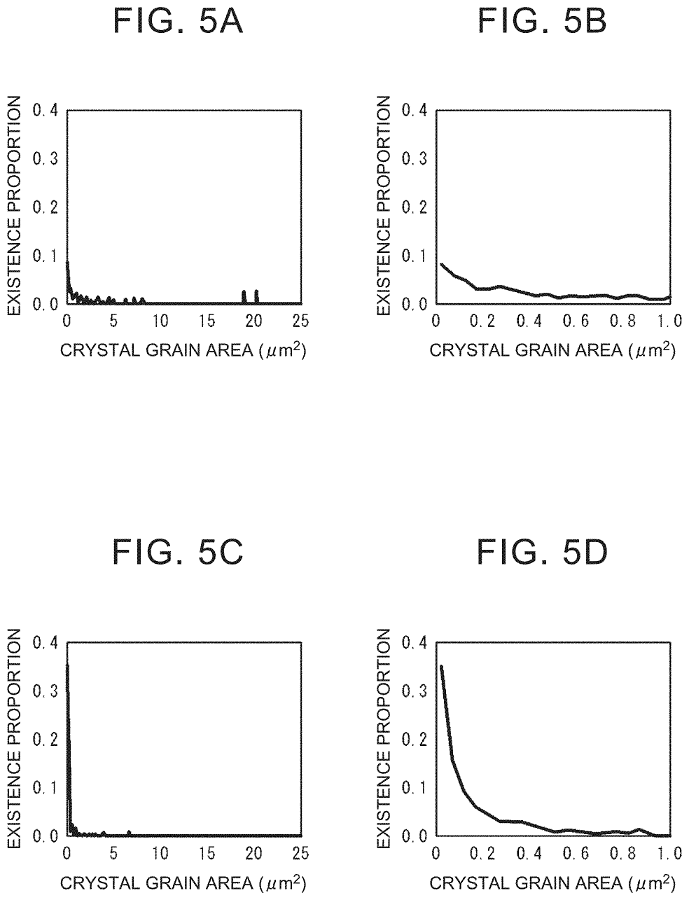

FIG. 5A to FIG. 5D show one example of histograms plotted based on the correspondence table. FIG. 5A is a view indicating the relationship between the crystal grain area before fatigue, and the relative frequency (existence proportion), and FIG. 5B is a view obtained by enlarging a range of the value of the crystal grain area from 0 to 1.0 μm² in FIG. 5A, while FIG. 5C is a view showing the relationship between the crystal grain area in the fatigue portion, and the relative frequency (existence proportion), and FIG. 5D is a view obtained by enlarging a range of the value of the crystal grain area from 0 to 1.0 μm² in FIG. 5C. FIG. 5A and FIG. 5B show values obtained using the inner ring 2 that has not been used, and FIG. 5C and FIG. 5D show values obtained using the inner ring 2 of which the calculation life ratio obtained in a durability test is 17.

As shown in FIG. 5A and FIG. 5B, the existence proportion of the crystal grains of the inner ring 2 before fatigue is relatively slightly large when the crystal grain area is in a range equal to or smaller than 0.2 μm², but spreads to such an extent that no large bias is observed, when viewed in a range equal to or smaller than 25 μm². On the other hand, the existence proportion of the crystal grains of the inner ring 2 after fatigue as shown in FIG. 5C and FIG. 5D assumes significantly large values when the crystal grain area is in the range equal to or smaller than 0.2 μm². From these graphs, it is found that there is a correlation between the existence proportion of particular crystal grains of which the crystal grain areas are within a predetermined numerical range, and the degree of fatigue. In this embodiment, the estimated degree of fatigue is obtained by utilizing the correlation between the existence proportion of the particular crystal grains and the fatigue degree.

The fatigue portion existence rate in the measurement area A1 is obtained based on the correspondence table shown in FIG. 4. The fatigue portion existence rate is the proportion of the particular crystal grains of which the crystal grain areas are within the predetermined numerical range present in the measurement area A1. In other words, the fatigue portion existence rate is the ratio of the number of the particular crystal grains, to the number of all crystal grains included in the measurement area A1. For example, when the predetermined numerical range of the crystal grain area used for determining the particular crystal grains is set to be larger than 0.0 μm² and equal to or smaller than 0.2 μm², crystal grains corresponding to the classes where the crystal grain area is equal to or smaller than 0.2 μm² are determined as particular crystal grains. Thus, the sum of the existence proportions in the respective classes where the crystal grain area is equal to or smaller than 0.2 μm² is obtained. The sum is the existence proportion of the particular crystal grains in the measurement area A1, and is the fatigue portion existence rate in the measurement area A1.

For example, when the fatigue portion existence rate in the case where the predetermined numerical range is larger than 0 μm², and is equal to or smaller than 0.2 μm² is obtained, based on the correspondence table shown in FIG. 4, the sum of the existence proportions corresponding to five classes of 0.025 μm², 0.075 μm², 0.125 μm², and 0.175 μm², which are surrounded by a frame H in the column of “FATIGUE PORTION” in FIG. 4, is obtained. In this case, the fatigue portion existence rate is 0.6521.

In this embodiment, the outside-fatigue-portion existence rate indicating the existence proportion of the particular crystal grains in the measurement area A2 is also obtained. Like the fatigue portion existence rate, the outside-fatigue-portion existence rate is obtained by a similar method, using the correspondence table shown in FIG. 4. In the manner as described above, in step S5 of FIG. 1, the fatigue portion existence rate in the measurement area A1 is obtained, and the outside-fatigue-portion existence rate in the measurement area A2 is obtained.

Then, as shown in step S6 of FIG. 1, the rate of change of the fatigue portion existence rate is obtained, and the degree of fatigue of the inner ring 2 is obtained, based on the change rate. The change rate of the fatigue portion existence rate is obtained based on the following equation.

Change rate of fatigue portion existence rate=((fatigue portion existence rate)−(initial value))/(initial value)

In the above equation, the initial value is the fatigue portion existence rate before fatigue of the fatigue portion. The initial value can be obtained by measuring the fatigue portion existence rate in advance, using an inner ring 2 cut before use, which was subjected to exactly the same production process as the inner ring 2 of which the degree of fatigue is to be estimated. However, it is difficult to obtain the initial value of the inner ring 2 as a product removed from the market. Thus, in this embodiment, the outside-fatigue-portion existence rate is used, in place of the initial value. As described above, in the bearing ring of the rolling bearing, rolling fatigue develops in a region inside the vicinity of the raceway surface on which the rolling element runs, and a sign of fatigue is not prominently seen in the surface portion. Thus, the outside-fatigue-portion existence rate can be regarded as substantially the same value as the fatigue portion existence rate before fatigue. Thus, in this embodiment, the change rate of the fatigue portion existence rate is obtained, using the outside-fatigue-portion existence rate, instead of the initial value. Thus, the initial value can be obtained, even where the product is removed from the market, and the change rate of the fatigue portion existence rate can be obtained with high accuracy.

Next, the estimated degree of fatigue of the inner ring 2 is obtained, based on the change rate of the fatigue portion existence rate. The estimated fatigue degree of the inner ring 2 is obtained, by referring to a fatigue database (database for use in fatigue estimation) created in advance.

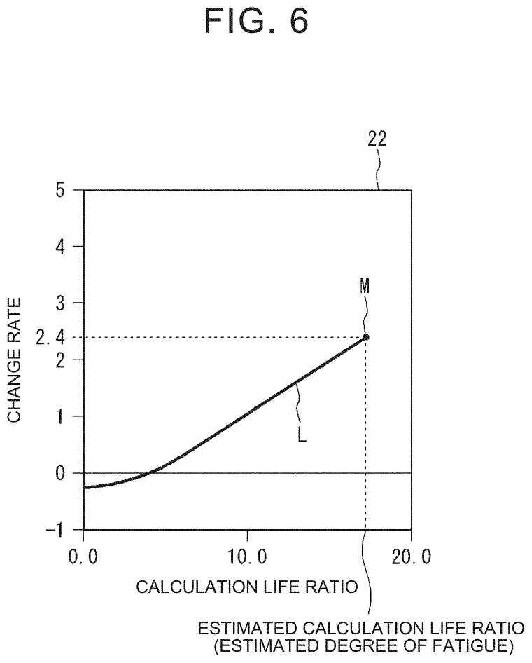

FIG. 6 shows the fatigue database in the form of a graph. The fatigue database 22 is data (numeral values or mathematical expressions) indicating the relationship between the change rate of the fatigue portion existence rate, and the fatigue degree of the inner ring 2. A method of creating the fatigue database 22 will be described later.

The fatigue database 22 shown in FIG. 6 indicates the relationship between the change rate of the fatigue portion existence rate and the fatigue degree when the numeral range of the crystal grain area which determines the particular crystal grains is equal to or smaller than 0.2 μm². The fatigue database 22 employs the calculation life ratio as the fatigue degree. The calculation life ratio is the proportion to the basic rating life L10 of the tapered roller bearing 1. The basic rating life L10 is the rating life when the reliability is 90%, under normal use conditions. The life mentioned herein denotes the total number of rotations of one bearing ring relative to the other bearing ring, until the initial sign of fatigue of a material appears in any of the bearing rings and rolling body of the bearing. The reliability denotes the ratio of the number of bearings that reach a particular life, or are expected to exceed the life, when a set of the same bearings is driven under the same conditions, to the total number of bearings. The rating life denotes a predicted value of the life based on a basic dynamic radial load rating, or basic dynamic axial load rating.

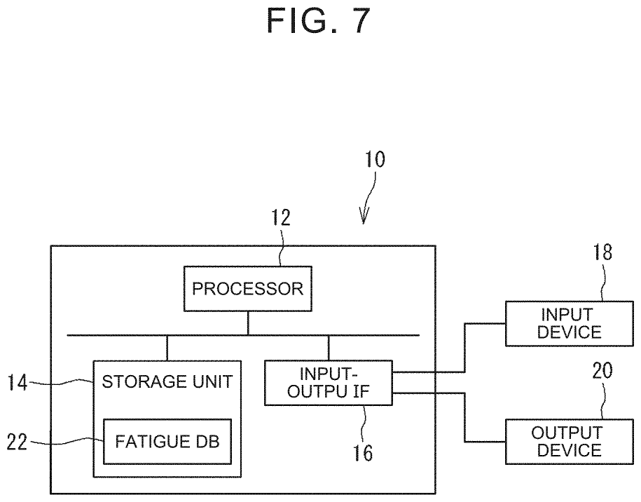

As indicated by the fatigue database 22 in FIG. 6, the change rate of the fatigue portion existence rate has a correlation to the calculation life ratio (fatigue degree). More specifically, the change rate of the fatigue portion existence rate has a generally linear relationship with the calculation life ratio. The fatigue database 22 is stored in a computing device for obtaining the estimated fatigue degree. FIG. 7 is a block diagram showing one example of the computing device for obtaining the estimated fatigue degree. The computing device 10 is constituted by a computer, or the like, including a processor 12 that consists of a central processing unit (CPU), etc., a storage unit 14 that consists of a hard disc, memory, etc., and an input-output interface 16. The storage unit 14 stores programs needed for operation of the computing device 10, various types of data, and so forth. The functions possessed by the computing device 10 are implemented when the processor 12 executes the programs stored in the storage unit 14. The processor 12 may implement the functions possessed by the computing device 10, by reading the programs recorded in a computer-readable recording medium or media.

To the input-output interface 16 are connected an input device 18 that consists of a keyboard, mouse, etc., and an output device 20 that consists of a display, printer, etc. The input-output interface 16 inputs and outputs various types of information, via the input device 18 and the output device 20.

As shown in FIG. 7, the fatigue database 22 is stored in the storage unit 14. When the change rate of the fatigue portion existence rate is given to the computing device 10 via the input device 18, the processor 12 obtains the calculation life ratio corresponding to the given change rate, referring to the fatigue database 22.

For example, when a value 2.4 is given as the change rate of the fatigue portion existence rate, to the computing device 10, the processor 12 obtains the calculation life ratio in the case where the value of the change rate is 2.4, referring to the fatigue database 22 (FIG. 6). The processor 12 specifies point M at which the value of the change rate is 2.4 in a graph L, in FIG. 6, and obtains the calculation life ratio corresponding to point M, as the estimated fatigue degree. In the case of FIG. 6, the calculation life ratio corresponding to the value 2.4 of the change rate is about 17. Thus, the processor 12 outputs information that the estimated calculation life ratio as the estimated fatigue degree of the inner ring 2 is 17, via the output device 20.

Thus, in this embodiment, the crystal grain areas are obtained using the crystal misorientations measured by the EBSD, so as to obtain the estimated degree of fatigue, thus permitting measurements in a more minute range than that of the X-ray diffraction method. Thus, the measurement area A1 can be precisely set for the fatigue portion in which fatigue develops, to permit measurements therein, and the crystal grain area of each crystal grain in the measurement area A1 can be obtained with high accuracy. Further, the fatigue portion existence rate and its rate of change obtained based on the crystal grain areas have correlative relationships with the calculation life ratio (fatigue degree) of the tapered roller bearing 1 made of a metallic material, and the estimated calculation life ratio can be thus obtained. With this configuration, it is possible to obtain the estimated degree of fatigue while assuring high accuracy in the measurement results in the fatigue portion, and enhance the accuracy in estimation of the fatigue degree.

Also, in this embodiment, the estimated degree of fatigue is obtained using the change rate of the fatigue portion existence rate; therefore, an influence due to a difference in the initial value of the fatigue portion existence rate before fatigue can be reduced, and the estimation accuracy can be further enhanced. Also, the change rate of the fatigue portion existence rate is obtained, based on the fatigue portion existence rate in the measurement area A1 set in the fatigue portion, and the outside-fatigue-portion existence rate in the measurement area A2 set as an area other than the fatigue portion. This makes it possible to obtain the change rate of the fatigue portion existence rate before and after fatigue of the tapered roller bearing 1, without preparing the tapered roller bearing 1 before fatigue in advance. Thus, it is possible to obtain the change rate of the fatigue portion existence rate, even with respect to a product recovered from the market.

Further, the surface portion in which the measurement area A2 is set in this embodiment has substantially the same metallographic structure as the fatigue portion before fatigue, and the outside-fatigue-portion existence rate in the surface portion can be considered as a value close to a value of the fatigue portion existence rate measured before fatigue develops in the fatigue portion. Thus, it is possible to obtain the change rate of the fatigue portion existence rate with improved accuracy, by using the outside-fatigue-portion existence rate in the surface portion.

In this embodiment, the estimated calculation life ratio is obtained by obtaining the fatigue portion existence rate (outside-fatigue-portion existence rate) from one measurement area A1 (A2). However, two or more calculation life ratios may be obtained from two or more measurement areas A1 (A2), and the estimated calculation life ratio may be obtained from the average of the obtained life ratios.

About Creation of Fatigue Database

Next, the method of creating the fatigue database 22 will be described. To create the fatigue database 22, a plurality of tapered roller bearings 1 is initially prepared, and a durability test is conducted on the tapered roller bearings 1, using a durability test machine. At this time, the durability test is conducted such that the calculation life ratios of the respective tapered roller bearings 1 spread over a range of about 0 to 17, for example. Thus, the tapered roller bearings 1 having different calculation life ratios (fatigue degrees) can be obtained.

Then, step S1 to step S6 of FIG. 1 are performed on each of the inner rings 2 of the tapered roller bearings 1 having different calculation life ratios. As a result, the fatigue portion existence rate, outside-fatigue-portion existence rate, and change rate of the fatigue portion existence rate can be obtained, with respect to each of the inner rings 2.

During execution of each step, the correspondence table as shown in FIG. 4 can be obtained with respect to each of the inner rings 2. Thus, when the fatigue portion existence rate and the outside-fatigue-portion existence rate are obtained, with respect to each of the inner rings 2, the numerical range of the crystal grain area for determining the particular crystal grains can be arbitrarily set. As a result, the fatigue portion existence rate, outside-fatigue-portion existence rate, and change rate of the fatigue portion existence rate can be obtained with respect to the particular crystal grains determined by the arbitrarily set numerical range of the crystal grain area.

Then, the calculation life ratio of each of the tapered roller bearings 1 is associated with the change rate of the fatigue portion existence rate of each of the inner rings 2 of the tapered roller bearings 1, so that the fatigue database 22 can be obtained. For example, where the numerical range of the crystal grain area for determining the particular crystal grains is set to be equal to or smaller than 0.2 μm², the fatigue database 22 as shown in FIG. 6 is obtained. The fatigue database 22 may be numerical data obtained by associating the calculation life ratio of each of the tapered roller bearings 1 with the change rate of the fatigue portion existence rate of each of the inner rings 2 of the tapered roller bearings 1, or may be an approximate expression that can be obtained based on the numerical data.

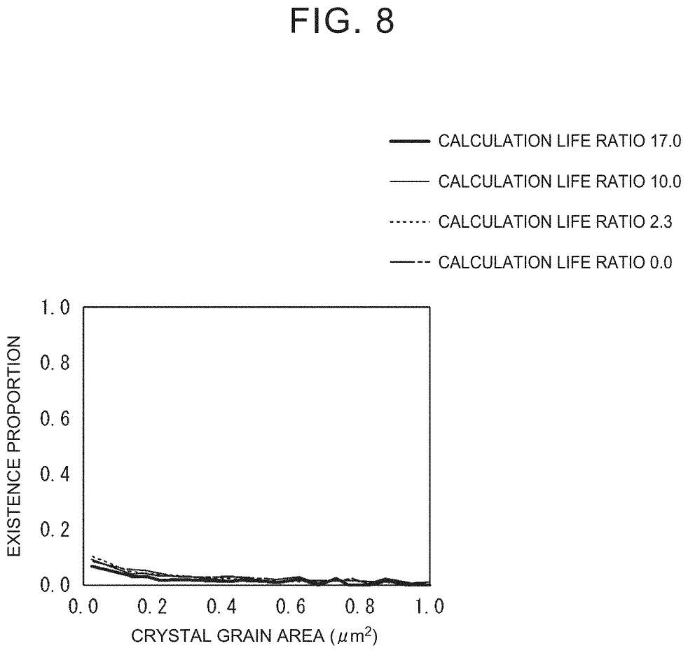

FIG. 8 shows a histogram obtained when the fatigue database 22 shown in FIG. 6 is created, and indicates the relationship between the crystal grain area (class) in the measurement area A2, and the existence proportion (relative frequency). FIG. 8 shows graphs representing four different calculation life ratios. As shown in FIG. 8, in any of the cases where the calculation life ratio is 0.0 (before fatigue), 2.3, 10.0, and 17.0, no significant change is seen in the distribution of the existence proportion against each crystal grain area. Namely, almost no change is seen in the distribution of the existence proportion in the measurement area A2, even if the calculation life ratio representing the fatigue degree increases. The inner ring 2 has some strain resulting from quenching, or the like, conducted in a manufacturing stage. FIG. 8 indicates that, even when the inner ring 2 is used, the outside-fatigue-portion existence rate almost keeps the initial value before fatigue. Namely, FIG. 8 confirms that the outside-fatigue-portion existence rate can be regarded as being substantially the same value as the fatigue portion existence rate before fatigue.

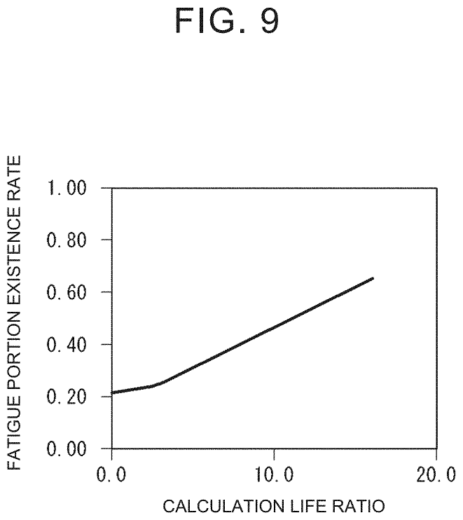

Like the change rate of the fatigue portion existence rate, the fatigue rate existence rate can also be associated with the calculation life ratio of each of the tapered roller bearings. FIG. 9 is a graph indicating the relationship between the fatigue portion existence rate and the calculation life ratio, which relationship is obtained when the fatigue database 22 shown in FIG. 6 is created. As shown in FIG. 9, like the change rate of the fatigue portion existence rate, the fatigue portion existence rate has a correlative relationship with the calculation life ratio (fatigue degree). Thus, the relationship between the fatigue portion existence rate and the calculation life ratio can be obtained as the fatigue database, and the estimated calculation life ratio can be obtained using the fatigue portion existence rate.

About Numerical Range of Crystal Grain Area Determining Particular Crystal Grains

In this embodiment, the lower limit and upper limit of the numerical range (predetermined numerical range) of the crystal grain area which determines the particular crystal grains may be arbitrarily set, but it is to be noted that crystal grains having relatively small crystal grain areas increase in proportion to fatigue, as shown in FIG. 5A to FIG. 5D. Thus, the numerical range of the crystal grain area which determines the particular crystal grains can be set to be equal to or smaller than a predetermined set value. Namely, the lower limit of the numerical range may be set to 0.0 μm², and the upper limit may be set to the set value. Thus, crystal grains having relatively small crystal grain areas, which increase in proportion to fatigue, can be included in the particular crystal grains.

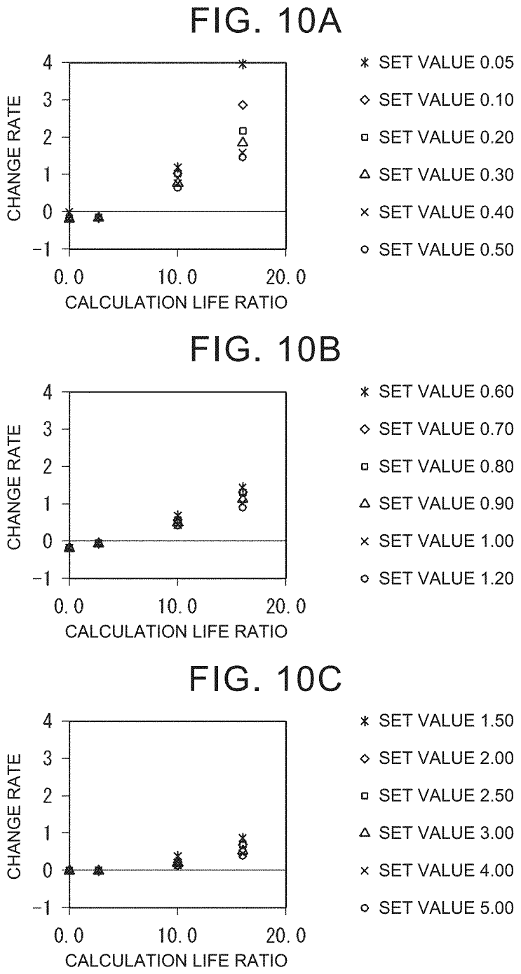

The set value will be described. FIG. 10A shows a fatigue database showing the relationship between the change rate of the fatigue portion existence rate and the calculation life ratio, when the set value is set within a range of 0.05 to 0.50 μm², and FIG. 10B shows a fatigue database showing the relationship between the change rate of the fatigue portion existence rate and the calculation life ratio when the set value is set within a range of 0.60 to 1.20 μm², while FIG. 10C shows a fatigue database showing the relationship between the change rate of the fatigue portion existence rate and the calculation life ratio when the set value is set within a range of 1.50 to 5.00 μm².

The fatigue databases of FIG. 10A to FIG. 10C are obtained by the same method as the above method of creating the fatigue database, and are obtained using the inner rings 2 of the tapered roller bearings 1. Also, the fatigue databases are created by plotting the change rates corresponding to the calculation life ratios 0, 2.3, 10, and 17.

As shown in FIG. 10A, when the set value is any one of 0.10 to 0.50 μm², the change rate of the fatigue portion existence rate increases almost monotonously relative to the calculation life ratio, which follows that there is a good correlative relationship between the change rate of the fatigue portion existence rate and the calculation life ratio. On the other hand, when the set value is 0.05 μm², the slope of a portion between a point where the calculation life ratio is 10 and a point where it is 17 is significantly larger than those of the other portions, and the correlative relationship between the change rate of the fatigue portion existence rate and the calculation life ratio is partially discontinuous. Thus, when the set value is 0.05 μm², the accuracy of the estimated calculation life ratio may be reduced.

Also, as shown in FIG. 10B and FIG. 10C, when the set value is any one of 0.60 to 5.00 μm², the change rate of the fatigue portion existence rate increases almost monotonously relative to the calculation life ratio, which follows that there is a good correlative relationship between the change rate of the fatigue portion existence rate and the calculation life ratio. Thus, when the calculation life ratio of the inner ring 2 is obtained based on the change rate of the fatigue portion existence rate, the set value is preferably equal to or larger than 0.10 μm².

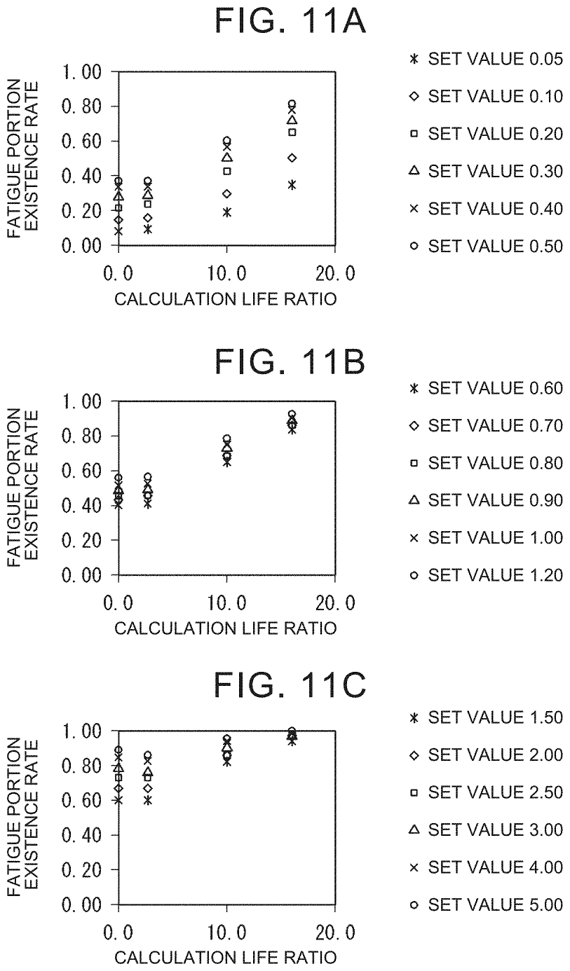

Next, the set value used when the fatigue database represents the relationship between the fatigue portion existence rate and the calculation life ratio will be described, using the same data as data used for creation of the fatigue databases shown in FIG. 10A to FIG. 10C. FIG. 11A shows a fatigue database indicating the relationship between the fatigue portion existence rate and the calculation life ratio when the set value is set within a range of 0.05 to 0.50 μm², and FIG. 11B shows a fatigue database indicating the relationship between the fatigue portion existence rate and the calculation life ratio when the set value is set within a range of 0.60 to 1.20 μm², while FIG. 11C shows a fatigue database indicating the relationship between the fatigue portion existence rate and the calculation life ratio when the set value is set within a range of 1.50 to 5.00 μm².

As shown in FIG. 11A and FIG. 11B, when the set value is any one of 0.05 to 1.20 μm², the fatigue portion existence rate increases almost monotonously relative to the calculation life ratio, which follows that there is a good correlative relationship between the fatigue portion existence rate and the calculation life ratio.

On the other hand, as shown in FIG. 11C, when the set value is any one of 3.00 to 5.00 μm², a portion where the fatigue portion existence rate does not monotonously increase relative to the calculation life ratio is seen when the calculation life ratio is equal to or smaller than 2.3, and the correlative relationship between the fatigue portion existence rate and the calculation life ratio is partially discontinuous. Thus, when the set value is in the range of 3.00 to 5.00 μm², the accuracy of the estimated calculation life ratio may be reduced.

Thus, when the calculation life ratio of the inner ring 2 is obtained based on the fatigue portion existence rate, the set value is preferably equal to or smaller than 2.5 μm². It follows that the set value is preferably equal to or larger than 0.1 μm², and is equal to or smaller than 2.5 μm². With the set value thus set within this range, a good linear relationship can be established between the change rate of the fatigue portion existence rate and the calculation life ratio, and between the fatigue portion existence rate and the calculation life ratio, and the estimated calculation life ratio can be obtained with high accuracy.

This disclosure is not limited to the embodiment illustrated above. In the illustrated embodiment, the computing device 10 performs operation to obtain the estimated calculation life ratio based on the change rate of the fatigue portion existence rate, in step S6 of FIG. 1. However, the computing device 10 may perform calculation of the crystal grain area, calculation of the fatigue portion existence rate, etc. in steps S4, S5 of FIG. 1. Further, a computer that controls the SEM and the EBSD may execute the method of this embodiment.

In the illustrated embodiment, the crystal grain area is obtained as the crystal grain size obtained based on the measurement result of the crystal misorientation. However, the average equivalent circle diameter of crystal grains may be obtained in place of the crystal grain area, and the fatigue estimation of the metallic material may be performed based on the average equivalent circle diameter.

In the illustrated embodiment, the outside-fatigue-portion existence rate in the measurement area A2 set in the surface portion is used as the initial value used when the change rate of the fatigue portion existence rate is obtained. However, the outside-fatigue-portion existence rate obtained by setting the measurement area A2 in a portion, such as a core portion of the inner ring 2, other than the surface portion and fatigue portion, may be used.

In the illustrated embodiment, the estimated calculation life ratio is obtained, using the change rate of the fatigue portion existence rate. However, the estimated calculation life ratio may be obtained using the fatigue portion existence rate, or may be obtained using both the change rate of the fatigue portion existence rate and the fatigue portion existence rate.

In the illustrated embodiment, the calculation life ratio is used as the degree of fatigue. However, a durability test may be conducted until damage arises from fatigue, and the degree of fatigue may be represented by a proportion to a test time from start of the test to a point in time when the damage arises, as a criteria (the maximum value).

While the fatigue degree of the inner ring of the tapered roller bearing is estimated in the illustrated embodiment, the fatigue degree may be estimated with respect to an outer ring or roller, or the estimated degree of fatigue may be obtained with respect to component parts of other rolling bearings, without being limited to the tapered roller bearing. Further, the method according to the disclosure is not limitedly applied to the rolling bearings, but may be applied to machine elements in which metal fatigue appears. In the illustrated embodiment, the estimated degree of fatigue is obtained with respect to a steel material, such as an alloy steel for machine structural use, or a carbon steel for machine structural use. However, the estimated degree of fatigue of a metallic material, such as an aluminum alloy, other than the steel materials may be obtained.

What is claimed is:

1. A fatigue estimating method of estimating a degree of fatigue of a metallic material, comprising:
estimating a fatigue portion in which fatigue appears in a section of the metallic material;
obtaining a crystal grain size of each of a plurality of crystal grains in a fatigue portion measurement area set in the fatigue portion, based on crystal misorientation in the fatigue portion measurement area;
obtaining a fatigue portion existence rate indicating an existence proportion of particular crystal grains of which the crystal grain size is within a predetermined numeral range, in the fatigue portion measurement area; and
obtaining an estimated degree of fatigue of the metallic material, based on at least one of the fatigue portion existence rate, and a change rate of the fatigue portion existence rate before and after fatigue of the metallic material.

2. The fatigue estimating method according to claim 1, further comprising:
obtaining the crystal grain size of each of the crystal grains in an outside-fatigue-portion measurement area set in a portion of the section of the metallic material other than the fatigue portion, based on the crystal misorientation in the outside-fatigue-portion measurement area; and
obtaining an outside-fatigue-portion existence rate indicating the existence proportion of the particular crystal grains in the outside-fatigue-portion measurement area,
wherein, when the estimated degree of fatigue of the metallic material is obtained, the change rate of the fatigue portion existence rate is obtained, based on the fatigue portion existence rate, and the outside-fatigue-portion existence rate.

3. The fatigue estimating method according to claim 2, wherein
the fatigue portion is a portion in which rolling fatigue appears, and a distance from a rolling contact surface of the metallic material to the fatigue portion in a depth direction is equal to or larger than a predetermined value; and
the portion other than the fatigue portion is a surface portion between the rolling contact surface of the metallic material and the fatigue portion.

4. The fatigue estimating method according to claim 1, wherein the predetermined numerical range is equal to or smaller than a predetermined set value.

5. The fatigue estimating method according to claim 4, wherein:
the crystal grain size is a crystal grain area; and
the predetermined set value is equal to or larger than 0.1 μm², and is equal to or smaller than 2.5 μm².

6. The fatigue estimating method according to claim 1, further comprising
creating a database indicating a relationship between at least one of the fatigue portion existence rate, and the change rate of the fatigue portion existence rate, and the degree of fatigue of the metallic material,
wherein the estimated degree of fatigue of the metallic material is obtained by referring to the database.

7. A method of creating a database for fatigue estimation of a metallic material, for use in the fatigue estimating method according to claim 1, the database being used when the estimated degree of fatigue of the metallic material is obtained, based on at least one of the fatigue portion existence rate, and the change rate of the fatigue portion existence rate, the method comprising:
obtaining a plurality of test pieces which have different degrees of fatigue, and are formed of the same material as the metallic material;
estimating a fatigue portion in which fatigue appears in a section of each of the test pieces;
measuring a distribution of misorientations in a fatigue portion measurement area set in the fatigue portion, with respect to each of the test pieces;
obtaining a crystal grain size of each of a plurality of crystal grains in the fatigue portion measurement area, based on the distribution of misorientations in the fatigue portion measurement area, with respect to each of the test pieces;
obtaining a fatigue portion existence rate indicating an existence proportion of particular crystal grains of which the crystal grain size is within a predetermined numerical range, in the fatigue portion measurement area, with respect to each of the test pieces; and
creating the database for fatigue estimation, by associating the degree of fatigue of each of the test pieces, with at least one of the fatigue portion existence rate of each of the test pieces and the change rate of the fatigue portion existence rate of each of the test pieces.
